(19) United States
(12) Patent Application Publication
(10) Pub. No.: US 2009/0093879 A1
(43) Pub. Date: Apr. 9, 2009

(54) MICRO- AND NANO-PATTERNED SURFACE FEATURES TO REDUCE IMPLANT FOULING AND REGULATE WOUND HEALING

(76) Inventors: Debra Wawro, Paradise, TX (US); Karen Pawlowski, McKinney, TX (US); Robert Magnusson, Arlington, TX (US); Peter Roland, Irving, TX (US); Roger Chan, Richardson, TX (US)

Correspondence Address: CARR LLP, 670 FOUNDERS SQUARE, 900 JACKSON STREET, DALLAS, TX, 75202, US

(21) Appl. No.: 12/245,702

(22) Filed: Oct. 4, 2008

Related U.S. Application Data
(60) Provisional application No. 60/977,606, filed on Oct. 4, 2007.

(51) Int. Cl. A61F 2/02

(52) U.S. Cl. 623/11.11

(57) ABSTRACT

An exemplary embodiment provides a medical implant with implant surfaces that are exposed to body tissue when the medical implant is inserted into a body of a recipient. The implant has a micro- or a nano-sized pattern on at least a portion of the implant surfaces. Optionally, the micro- or nano-sized pattern may be a periodic (or “repeating”) pattern. Further, the micro- or nano-sized pattern may have geometric features, such as grooves, circles, triangles, rectangles, pentagons and hexagons.

### STATEMENT OF RELATED APPLICATIONS

This application relates to and claims the benefit of the filing date of U.S. provisional application Ser. No. 60/977,606 entitled MICRO- AND NANO-PATTERNED SURFACE FEATURES TO REDUCE FOULING AND REGULATE WOUND HEALING filed Oct. 4, 2007, the entire contents of which are incorporated herein by reference for all purposes.

### BACKGROUND

1. Technical Field

Various aspects and embodiments relate generally to medical implants that have micro- or nano-patterned surfaces.

2. Description of the Related Art

The field of medical implants is growing rapidly. For example, the number of cochlear implant recipients in 1995 was estimated to be about 12,000. Now, the number approaches 60,000, including more than 10,000 children with the Nucleus® trademark of Cochlear Americas Centennial, CO) device. Further, the upward trend is also reflected in other medical implants, such as hip and knee replacement implants. Implant-related medicine and technology has also evolved over recent years to the point that some implants are expected to remain inside the body for the majority of the recipient's lifetime.

Medical implants may be made of a variety of materials that are selected based on several criteria. These criteria may include, for example, particular properties that are well-suited to the service that the medical implant would be expected to perform. For example, knee and hip replacement implants are load bearing and as a result materials may be selected based on modulus and non-toxicity, among other criteria. Metal alloys are frequently selected to meet these criteria. In other medical implants, such as breast implants, materials may include a suitable inert material such as medical grade silicone gel or may be a combination of a silicone elastomer shell that is filled with a saline solution. In general, materials for other medical implants, such as cochlear implants, catheters, stents, pacemakers, and tympanostomy tubes, are also selected for particular physical properties so that they are able to withstand ordinarily expected conditions of use within the recipient's body. Materials for medical implants may include metal alloys, plastics, ceramics and combinations of these. For example, an acetabular cup of a hip replacement implant may have a metal alloy outer shell that has an interior surface lined with a high density polymer.

### SUMMARY

An exemplary embodiment provides a medical implant with implant surfaces that are exposed to body tissue when the medical implant is inserted into the body of a recipient. The implant has a micro- or a nano-sized pattern on at least a portion of the implant surfaces. Optionally, the micro- or nano-sized pattern may be a periodic (or “repeating”) pattern. Further, the micro- or nano-sized pattern may have geometric features, such as grooves, circles, triangles, rectangles, pentagons, hexagons and the like. Furthermore, the groove cross section profile can be sinusoidal, rectangular, trapezoidal, cylindrical and the like.

Another exemplary embodiment provides a medical implant that has implant surfaces exposed to body tissue when the medical implant is inserted into a body of a recipient. At least a portion of the implant surfaces has a micro- or a nano-sized pattern that controls and/or modifies micro-organism or fibroblast adhesion to the implant surfaces.

Yet another exemplary embodiment provides a medical implant of a biocompatible material that has surfaces exposed to body tissue and fluid when the medical implant is surgically implanted into the body of a recipient. At least a portion of the surfaces have a micro- or nano-sized pattern. Optionally, the micro- or nano-sized pattern may be of a biocompatible material different from the biocompatible material of the medical implant. In an exemplary embodiment, the micro- or nano-sized pattern may be silica. The medical implant may be selected from knee implants, hip implants, cardiac stents, cochlear implants, catheters, pacemakers, breast implants, and tympanostomy tubes, for example.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is an illustration of the relevant features of a periodic pattern with a rectangular groove cross-section. Features include the pattern period, the groove depth, the groove peak width (or fill factor) and the remaining groove valley width (1 minus the fill factor);

FIG. 1A is an illustration of the relevant features of a periodic pattern with a trapezoidal groove cross-section. Features include the pattern period, the groove depth, the groove peak width (or fill factor) and the remaining groove valley width (1 minus the fill factor);

FIG. 2 is an illustration of an atomic force micrograph of a grooved pattern with a 10 micron period, a 0.5 fill factor with and a ˜775 nm groove depth according to an exemplary embodiment;

FIG. 3 is an illustration of an atomic force micrograph of a grooved pattern with ˜255 nm groove peak width, and a 510 nm period, according to an exemplary embodiment;

FIG. 4 Fluorescent (A) and transmitted light (B) micrographs of a micro-patterned silicone surface (period=4.8 microns, features depicted in FIG. 8) demonstrating the collection of S. aureus bacteria and biofilm formation along the grooves of the patterned silicone;

FIG. 5 depicts fibroblasts on a blank (i.e. un-patterned) silicone surface, as a control;

FIG. 6 depicts fibroblasts on a nano-patterned silicone surface showing the uniform orientation of the fibroblasts aligned along the pattern, according to an exemplary embodiment;

FIG. 7 depicts fibroblasts aligned substantially uniformly on a micro-patterned surface, according to an exemplary embodiment;

FIG. 8 is a depiction of a portion of a surface of an exemplary embodiment having surface features with ˜4.8 microns period, ˜775 nm in groove depth and a ˜0.8 fill factor;

FIG. 9 is a depiction of a portion of a surface of an exemplary embodiment having surface features with a 510 nm periodicity, 0.6 fill factor and ˜200 nm groove depth;

FIG. 10 is an SEM depicting the formation of an S. aureus bio-film on unpatterned silicone in culture;

FIG. 11 is an SEM showing wild S. aureus on an unpatterned silicone surface of a cochlear implant receiver/stimulator device, removed from a patient due to intractable infection;

FIG. 12 is an SEM of wild S. aureus on another on an unpatterned cochlear receiver/stimulator device after preservation of the bio-film matrix with arrows indicating inflammatory cells incorporated within the bio-film;

FIG. 13 is a higher magnification image of FIG. 12, showing a three dimensional structure of the bio-film;

FIG. 14 is an SEM of fibroblasts cultured on micro-patterned surfaces (surface features described in FIG. 8), according to an exemplary embodiment;

FIG. 15 is an SEM depicting sheets of the fibroblasts formed on a micro-patterned surface (surface features described in FIG. 8) in accordance with an exemplary embodiment;

FIG. 16 is a light micrograph showing fibroblasts covering a portion of the surface on an unpatterned, polystyrene culture dish. The fibroblasts are numerous and randomly oriented;

FIG. 17 is a light micrograph showing fibroblasts grown on an unpatterned silicone disk that was cultured within the same dish used in FIG. 16. Fewer fibroblasts are seen on the silicone surface compared to the polystyrene surface of the culture dish;

FIG. 18 shows fibroblasts grown on a micro-patterned surface of a silicone disk (features described in FIG. 8). This patterned silicone disk was cultured within the same dish used in FIG. 16. The fibroblasts assemble in an organized manner compared to the unpatterned surfaces in FIGS. 16 and 17;

FIG. 19 shows fibroblasts shows fibroblasts grown on a nano-patterned surface of a silicone disk (features described in FIG. 9). This patterned silicone disk was cultured within the same dish used in FIG. 16. The fibroblasts assemble in an organized manner compared to the unpatterned surfaces in FIGS. 16 and 17;

FIG. 20 shows a picture of a typical (unpatterned) cochlear receiver/stimulator that is implanted under the scalp in implant patients. The electronics are embedded in a shell of silicone material;

FIG. 21 shows a picture of a typical (unpatterned) cochlear implant electrode that is connected via a silicone-coated wire to the receiver/stimulator shown in FIG. 20. The implant electrode is embedded in a silicone cover;

FIG. 22 is an SEM of a sheet of fibroblasts cultured on a blank control surface;

FIG. 23 is an SEM of a sheet of fibroblasts cultured on a nano-patterned surface (features described in FIG. 9), according to an exemplary embodiment;

FIG. 24 depicts a cochlear implant receiver having a portion having a micro and/or nano patterned surface;

FIG. 25 depicts a breast implant having a portion having a micro and/or nano patterned surface; and

FIG. 26 depicts a micro pattern surface in perspective view, having a nano pattern superimposed thereon.

### DETAILED DESCRIPTION

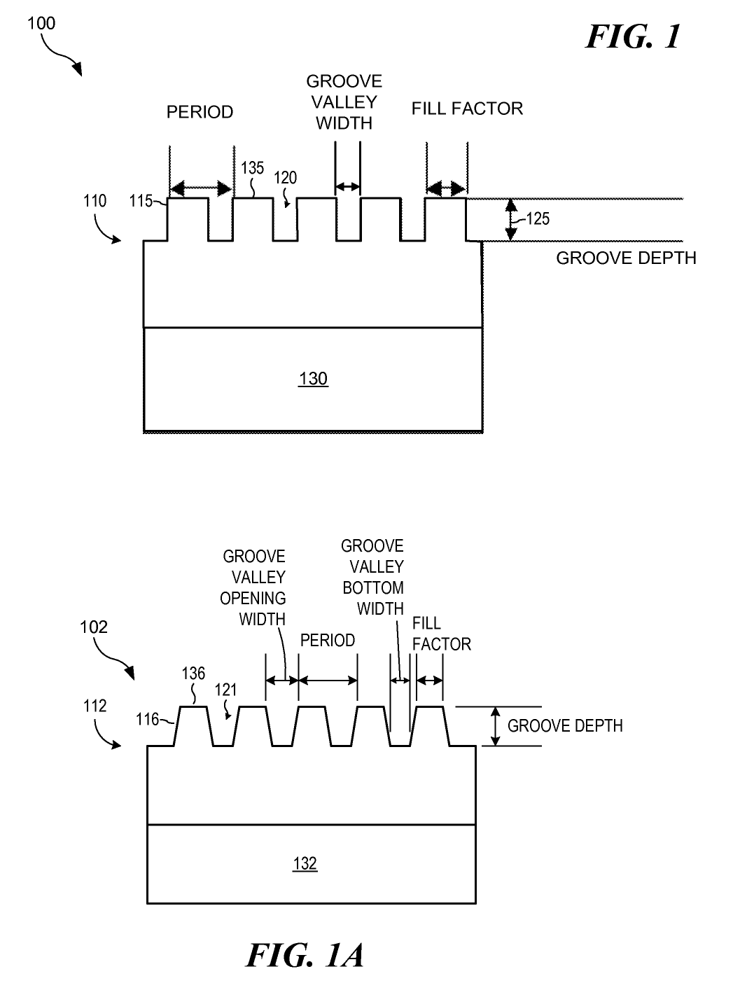

An exemplary embodiment provides medical implants that have at least partially micro-patterned or nano-patterned surfaces, as depicted in FIG. 1. FIG. 1 shows an illustration of the relevant features of surface 100 with a periodic pattern with a rectangular groove cross-section. Features include the pattern period, the groove valley 120, which has a depth with sides 130, the groove peak 135, which has a width (or fill factor), the remaining groove valley 120 having a width (i.e., 1.0 minus the factor). The patterned surface can be a coating 110 on a substrate 130, or can be integral with the substrate.

FIG. 1A is an illustration of the relevant features of a periodic pattern, similar to FIG. 1, but with a trapezoidal groove cross-section. Features include the pattern period, the groove valley 121, which has a valley bottom width, a valley opening width and a depth with sides 116, and the groove peak 136, which has a width. The grooves and trapezoids each have an average width, which, added together, would equal the period. For purposes of definition, in the case of a trapezoid (or other shape not having vertical sides, such as a sinusoid or triangle), the “fill factor” will be defined as the width of the trapezoid or other shape at the mid-height of the trapezoid or other shape. The patterned surface can be a coating 112 on a substrate 132, or can be integral with the substrate. Of course, other cross-sectional shaped can be used, such as sinusoidal, triangular, Aztec-shaped, asymmetric or other.

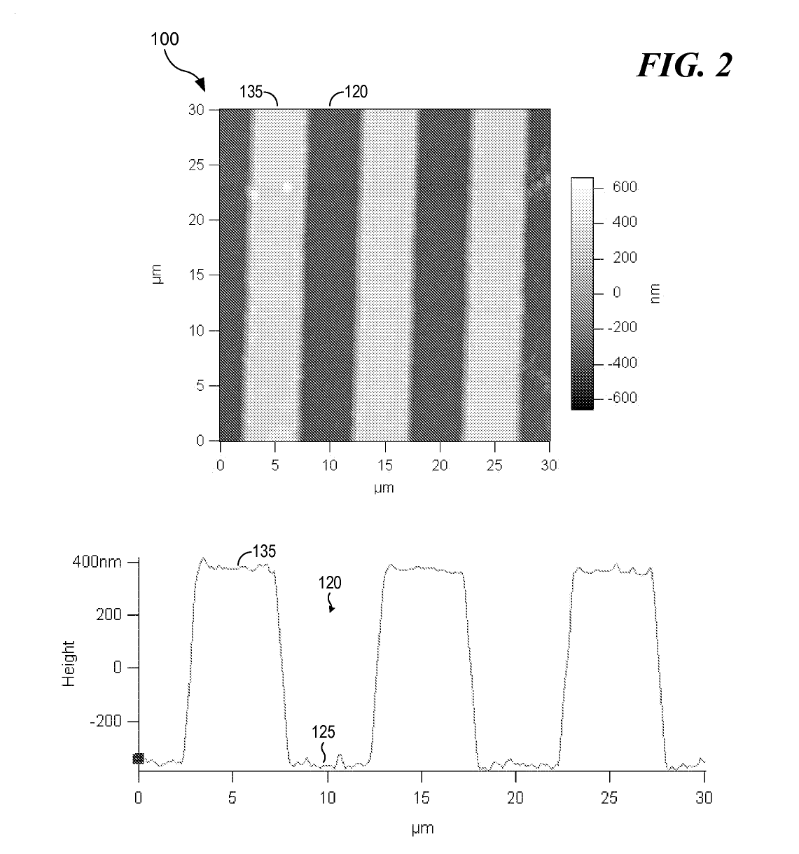

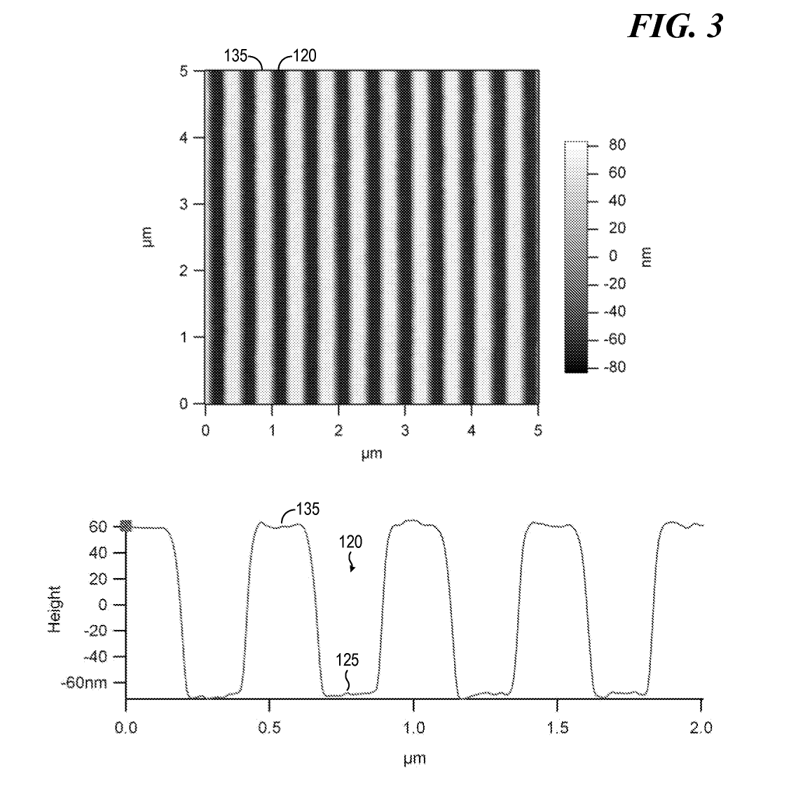

The pattern may be replicated at intervals to form a periodic or repeating pattern. The micro- or nano-sized patterns may be regular or random. The patterns include “features” that are arranged into a pattern. These features may have any of a variety of shapes, including for example, grooves, geometric shapes, and the like. The features of the micro- or nano-sized patterns may be in relief on the medical implant surface but may also be impressed into the surface. For example, as shown in FIG. 2, the features form a 10 micron periodic pattern, with groove widths of approximately 5 microns, and groove depths of 775 nm. In FIG. 3, the features form a 510 nm periodic pattern, with a groove width of approximately 255 nm, and groove depths of approximately 100 nm. The extent of the nano-sized patterns include groove depths of 1 nm to about 2 microns, groove fill factors from 0.01 to 0.99, and feature periodicities of 50 nm to 800 nm. For non-periodic patterns, feature sizes range from 1 nm to 400 nm in dimension.

In the context of the present invention, a 50% fill factor means that the peaks are 50% and valleys are 50% of the cross-section of the pattern. 80% fill factor means that the peaks are 80% of the grating period, with the remaining valleys being 20% of the period (i.e., 1.0-0.8=0.2 or 20%).

For example, a micro pattern having a 50% fill factor can have grooves that have a micro-sized groove valley width between about 1 to 3 times the width of a biofilm-forming bacteria (or other biofilm forming organism, such as a fungus) to be inhibited. Such an organism can closely fit within a groove, adjacent grooves being separated by a ridge having a groove depth about 0.5 to 5 times the width of that organism, and a groove peak width about 0.25 to 3 times the width of that organism. Of course, other fill factors could be used. For micro-sized patterns, 1 to 20 micron sized periodicities can be used, with grooves and peaks can have dimensions ranging from about 500 nm to 15 microns in width are desirable.

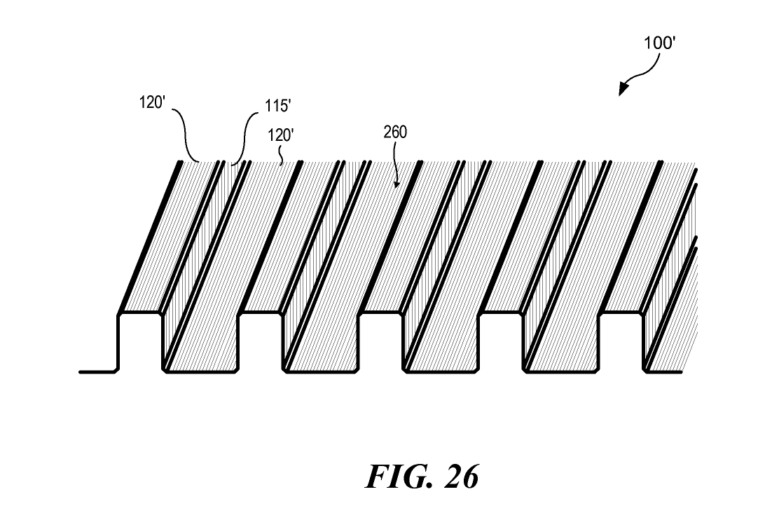

Micro and nano sized patterns can also be combined in the same structure 110′, as depicted in FIG. 26. The surface of at least the bottom of the micro-sized grooves 120′, and preferably also the side walls 115′ of the groove and the top of the peak 135′ of the micro-sized grooves, can be provided with nano-sized periodicities that can be many times smaller than the micro-sized grooves or peaks, and many times smaller than the organism(s) to be inhibited. For example, a 10 micron wide peak (and micro-groove bottom) can each have approximately five or more nano-sized grooves 260 superimposed on them. It is believed that the nano-sized period grooves can inhibit attachment of the organism by inducing changes in the surface properties (including, for example, surface energy, surface tension, wettability, hydrophobic/hydrophilic forces, surface charge) of the surfaces of the much larger micro-sized grooves and peaks, while the larger, micro-sized grooves (that the organism can fit into) can reduce the density and extent of the film formed by organisms that do attach despite the nano-sized grooves, by interrupting the ability of the organisms in the grooves to communicate colony-forming information with organisms in adjacent grooves, or to organisms that are not their immediate neighbors within a single groove, thus inhibiting the ability of the organisms to communicate and transfer resistance capabilities through the extracellular matrix (i.e, by quorum sensing).

Another exemplary embodiment provides medical implants that have surfaces at least partially micro-patterned and/or nano-patterned wherein the features are closely-spaced micro- or nano-sized grooves that are substantially parallel. These grooves may be formed between closely spaced walls or ridges that may be imprinted on the implant surfaces or may be impressed into the surfaces. The closely-spaced grooves may cover the implant surface. Alternatively, a group of grooves may form a pattern. This pattern may be repeated at predetermined intervals to form an overall pattern. In other words, the micro- or nano-grooved pattern may be “periodic,” meaning that it includes a repeating series of groups of grooves. Of course, other patterns of features other than grooves may also be periodic.

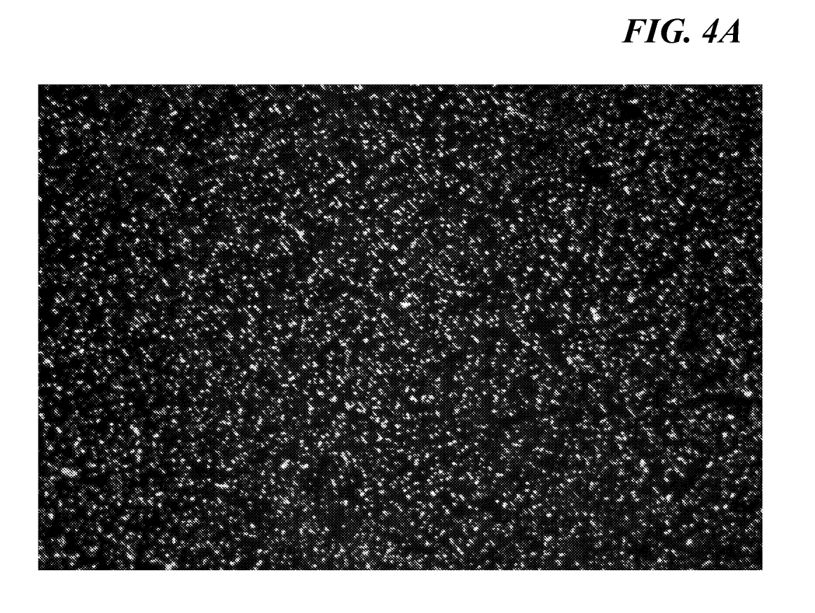

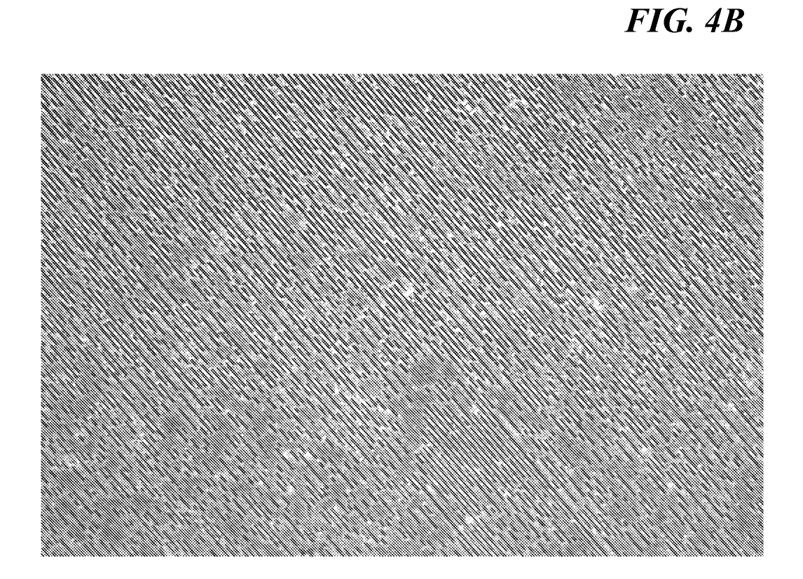

An exemplary embodiment reduces bio-film formation on the medical implant surface when the micro- or nano-patterned surface is implanted in the recipient and the surface is exposed to conditions within the body of the recipient. It is theorized, without being bound, that the micro- or nano-patterned surface either prevents formation of a bio-film, or disrupts, or impairs the integrity of a bio-film that may form on implant surfaces. Bio-film reduces the efficacy of antibiotics. As a consequence of the embodiment, infections are more readily treated with antibiotic therapy, and wound healing may be facilitated. In addition, infection rates may be reduced when bio-film prevention is effective. An example of an embodiment is depicted in FIG. 4, where the surface has a grooved micro-pattern. As shown, the bacteria, S. aureus grow within the grooves, resulting in a disruption of the biofilm and quorum-sensing communication between the bacteria across the grooves. As a result, the micro-grooves prevent or disrupt the formation of a uniform bio-film that covers the surface.

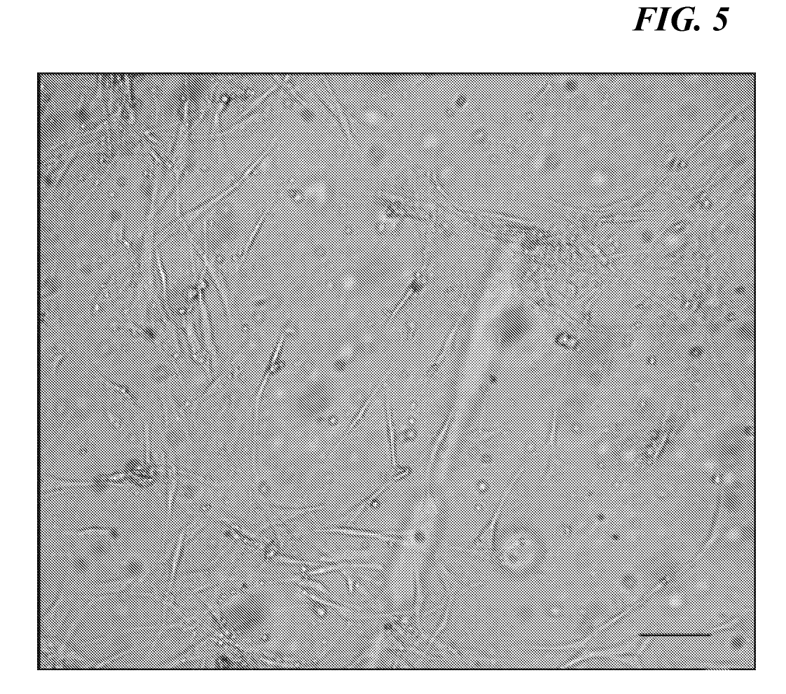

An exemplary embodiment of medical implants with surfaces that are micro- or nano-patterned to control or modify the adhesion of fibroblasts and thereby promote wound healing. In general, medical implants may trigger a range of adverse reactions in a recipient. These may include inflammation of tissue around the implant, and encapsulation of the implant with fibrocytes. It is theorized, without being bound, that micro- or nano-patterning of the implant surface provides contact guidance to fibrocytes and other cells associated with wound healing so that these migrate to the implant surfaces in a more ordered manner. Consequently, the formation of thick, fragile fibrotic scars is minimized and wound healing is promoted. Because fibrotic wounds have a limited capability to clear infections, reducing fibrosis also minimizes the risk of infection. An example compared with a control is illustrated in FIGS. 5-7.

FIG. 5 depicts fibroblasts on a blank (i.e. un-patterned) silicone surface used as a control. The fibroblasts are randomly oriented.

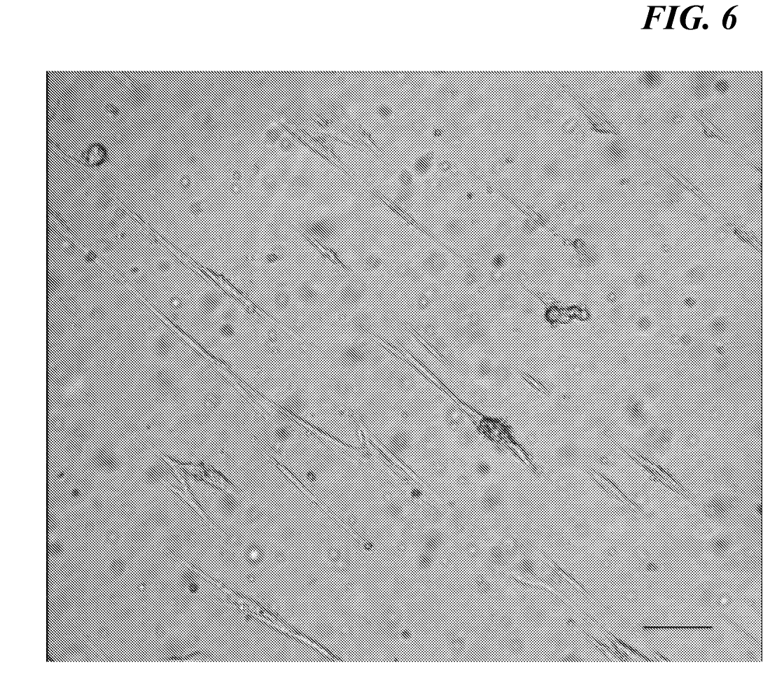

FIG. 6 depicts fibroblasts on a nano-patterned silicone surface showing the uniform alignment of the fibroblasts along the grooved pattern.

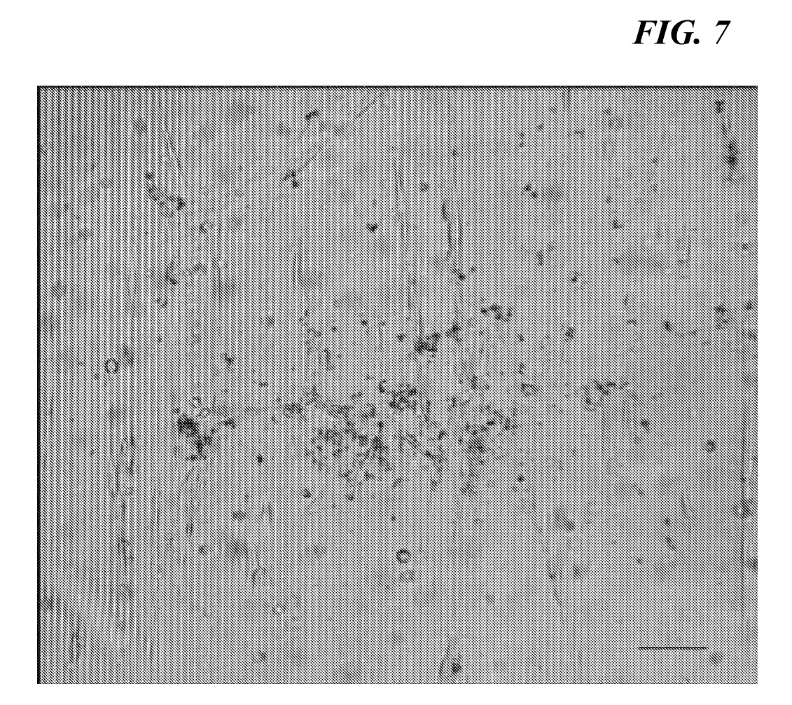

FIG. 7 depicts fibroblasts on a micro-patterned surface. These fibroblasts are also substantially aligned along the micro-groove pattern of the surface. Accordingly, these exemplary embodiments control or modify fibroblast adhesion.

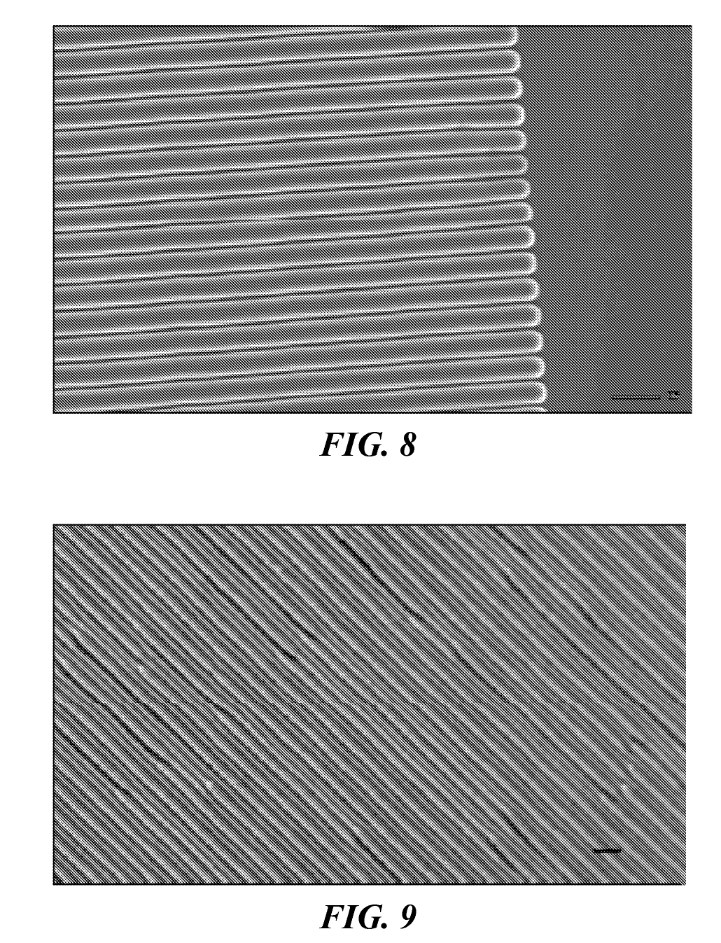

A further exemplary embodiment provides medical implants that have micro- or nano-sized non-random patterns covering at least a portion of the implant surfaces. The non-random patterns may include groups of any geometric shapes, for example, grooves, circles, triangles, rectangles, hexagons, pentagons, and the like. The non-random pattern may be a continuous or a periodic pattern covering at least a portion of the surfaces of the implant. FIG. 8 depicts an exemplary embodiment that has a periodicity of 4.8 microns, with features shown as parallel grooves. FIG. 9 is another exemplary embodiment that has a feature periodicity of 510 nm, with groove feature sizes of approximately 255 nm also shown as parallel grooves.

Exemplary embodiments of micro- or nano-patterned medical implants may have patterns of the same or different material of the medical implant to which the surface patterning is applied. The surface patterning materials may be selected to be compatible with the implant material (e.g. adherent to the implant material). But, the materials may also be selected to promote another useful physical property. For example, the surface patterning material may be used to tailor the surface energy or hydrophobic properties compared to the unpatterned implant surfaces. For example, by applying a surface pattern to the outside of cochlear implant electrode or deep brain electrode, the hydrophobic properties might be changed such that the implant is less likely to stick to surrounding tissue and can more easily glide into place, thus reducing physical damage to surrounding tissue during insertion. FIG. 21 illustrates a typical cochlear implant electrode.

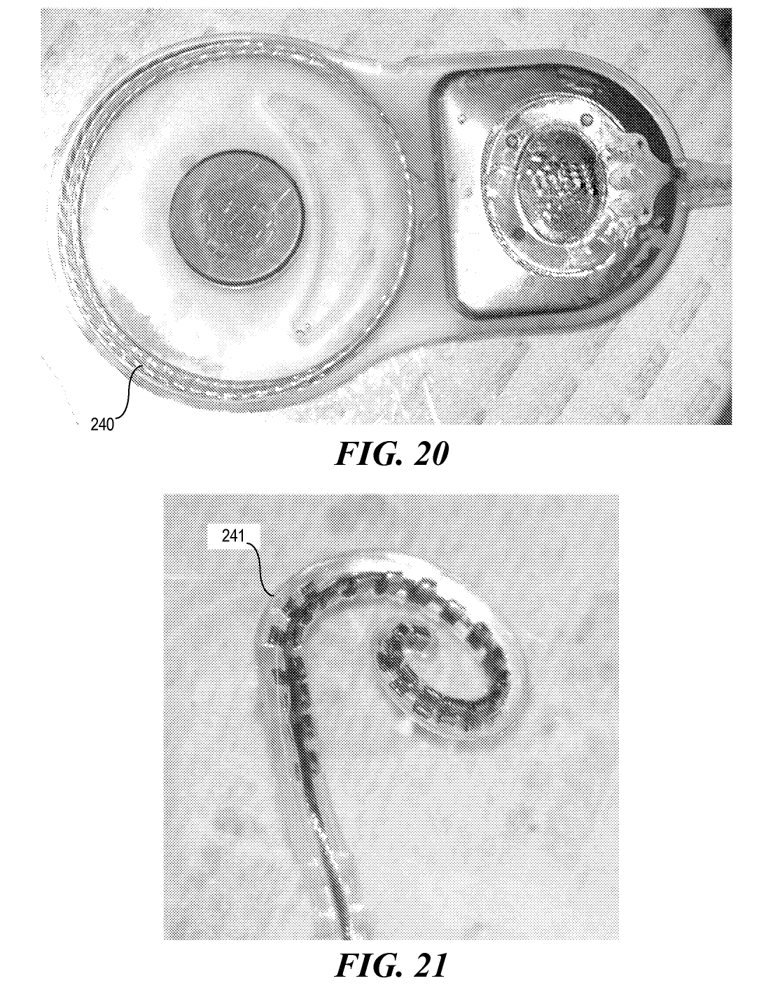

An exemplary embodiment provides silicone medical implants with surfaces that have been micro- or nano-patterned by contact printing of the uncured silicone elastomer surfaces. Silicone is widely used in cochlear implants and breast implants, among others. FIGS. 20 and 21 illustrate typical cochlear receiver/stimulator 240 and electrode 241 implants.

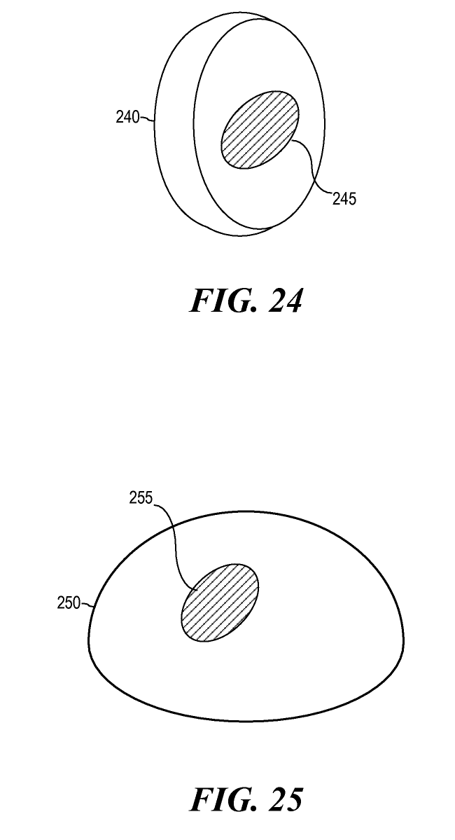

FIG. 24 depicts a cochlear implant receiver 240 having a portion 245 having a micro and/or nano patterned surface. FIG. 24 depicts the implant receiver 240 as having only a portion 245 of the surface being micro or nano patterned for purposes of simplicity of the FIGURE. However, it should be noted that preferably all or most of the surface of the implant receiver 240 will have a micro and/or nano patterned surface, dimensioned in accordance with the teachings of this invention for reducing biofilm attachment and/or for organizing fibroblasts.

FIG. 25 depicts a breast implant having a portion having a micro and/or nano patterned surface. FIG. 25 depicts the implant 250 as having only a portion 255 of the surface being micro or nano patterned for purposes of simplicity of the Figure. However, it should be noted that preferably all or most of the surface of the implant 250 will have a micro and/or nano patterned surface, dimensioned in accordance with the teachings of this invention for reducing organized biofilm attachment and/or for orienting fibroblasts.

In an exemplary method, surface patterning may be carried out by photolithographic techniques. A master pattern may be created on a silicon or quartz wafer or mold for the implant, using, for example, techniques of semiconductor manufacture, and the master pattern may then be used to contact print replicated patterns onto an implant surface, before or after molding the implant.

### EXAMPLES

### Influence of Micro- or Nano-Patterning on Staphylococcus aureus

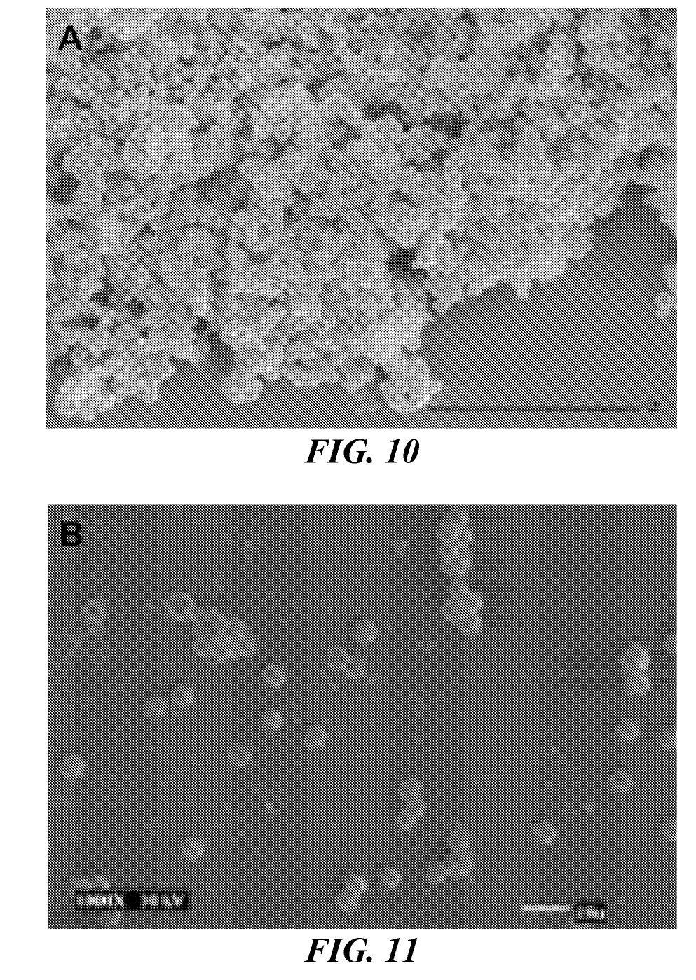

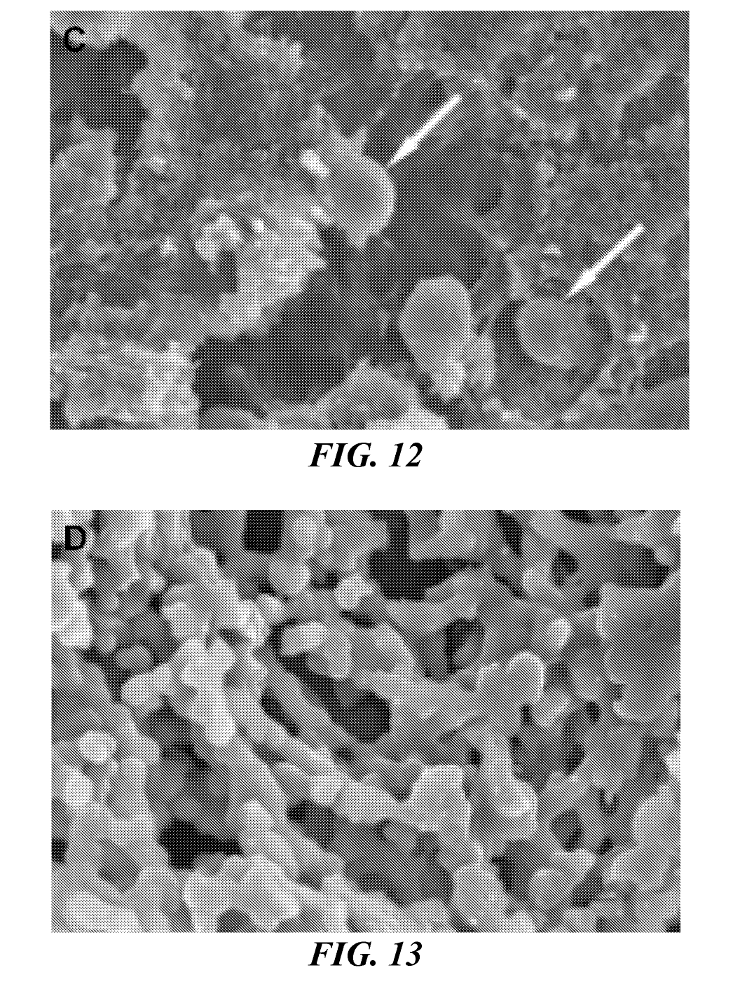

A sample of non-surface-patterned silicone was prepared in the lab. FIG. 10 is an SEM depicting the formation of an S. aureus bio-film on the unpatterned silicone in culture. In comparison, FIG. 11 is an SEM showing wild S. aureus on a silicone surface of a cochlear implant, removed from a patient due to intractable infection. FIG. 12 is an SEM of wild S. aureus on another cochlear device after preservation of the bio-film matrix. Arrows indicate inflammatory cells incorporated within the bio-film. FIG. 13 is a higher magnification image of FIG. 12 showing a three dimensional structure of the bio-film. These images illustrate S. aureus biofilms can form on silicone implant material in the body and in our culture model.

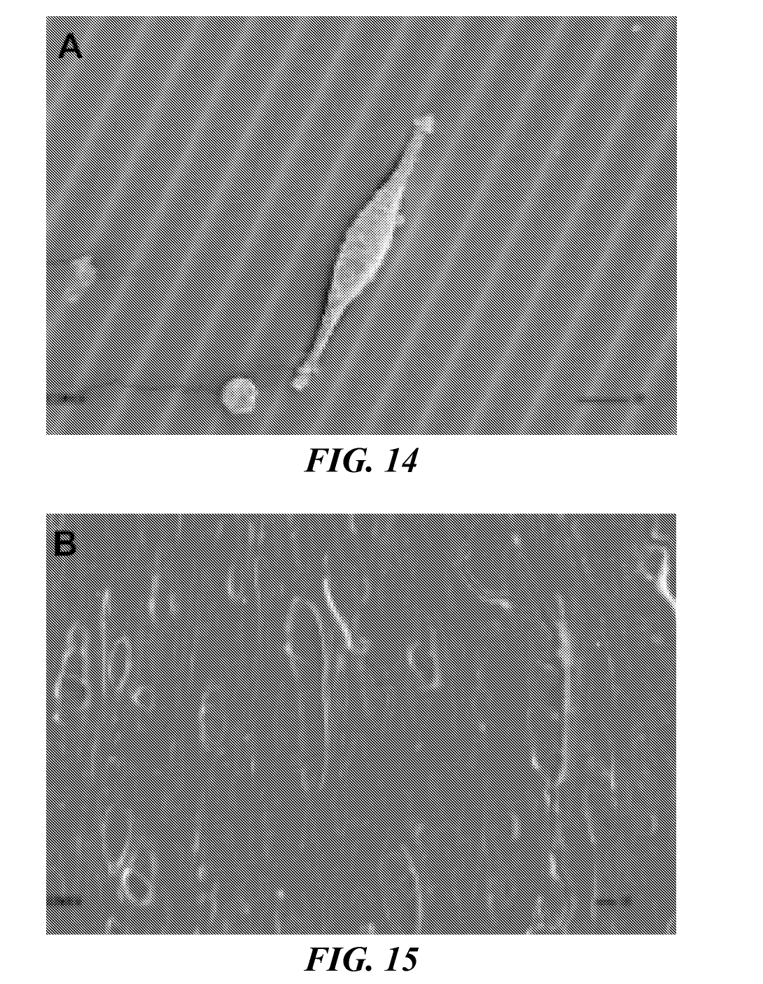

FIG. 14 is an SEM of fibroblasts cultured on micro-patterned-surfaces. Initially, the fibroblasts tend to line up along the direction of the grooved pattern. Eventually, as shown in FIG. 15, the fibroblasts formed sheets of cells that laid down in an organized fashion. This also occurred on the nano-patterned silicone surfaces.

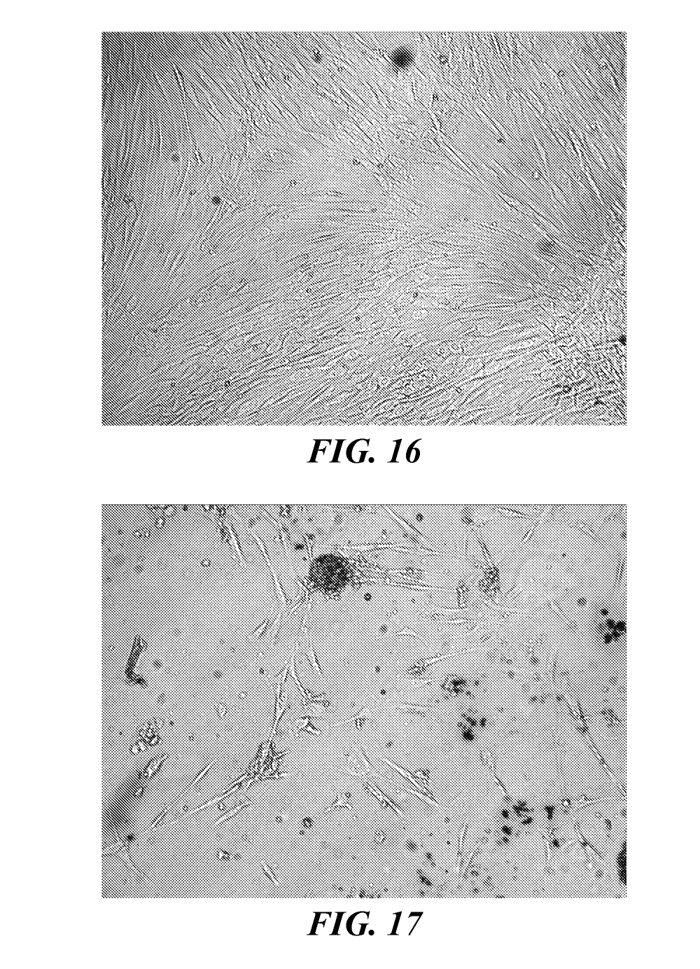

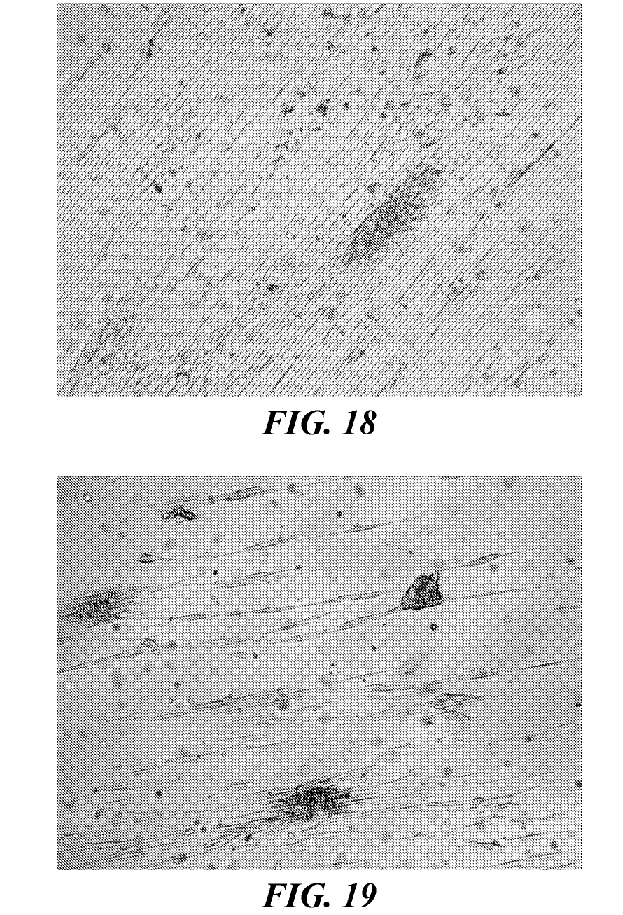

FIGS. 16-19 are light micrographs of fibroblasts cultured in the same dish that contained molded silicone discs. Although more fibroblast grew on the surface of the dish (FIG. 16) than on the silicone discs (FIGS. 17-19), the fibroblasts could attach and grow across even the un-patterned silicone. The organization of fibroblasts was random (un-patterned) dish surface (FIG. 16) and the un-patterned silicone (FIG. 17). The fibroblasts growing across the micro-patterned (FIG. 18) and the nano-patterned (FIG. 19) surfaces orient parallel to the lines and grooves.

FIGS. 20-21 are pictures of typical cochlear implant devices, including the receiver/stimulator (FIG. 20) and the accompanied cochlear implant electrode (FIG. 21). Both implants are integrated in and around a silicone mold. In these pictures, these implants are unpatterned.

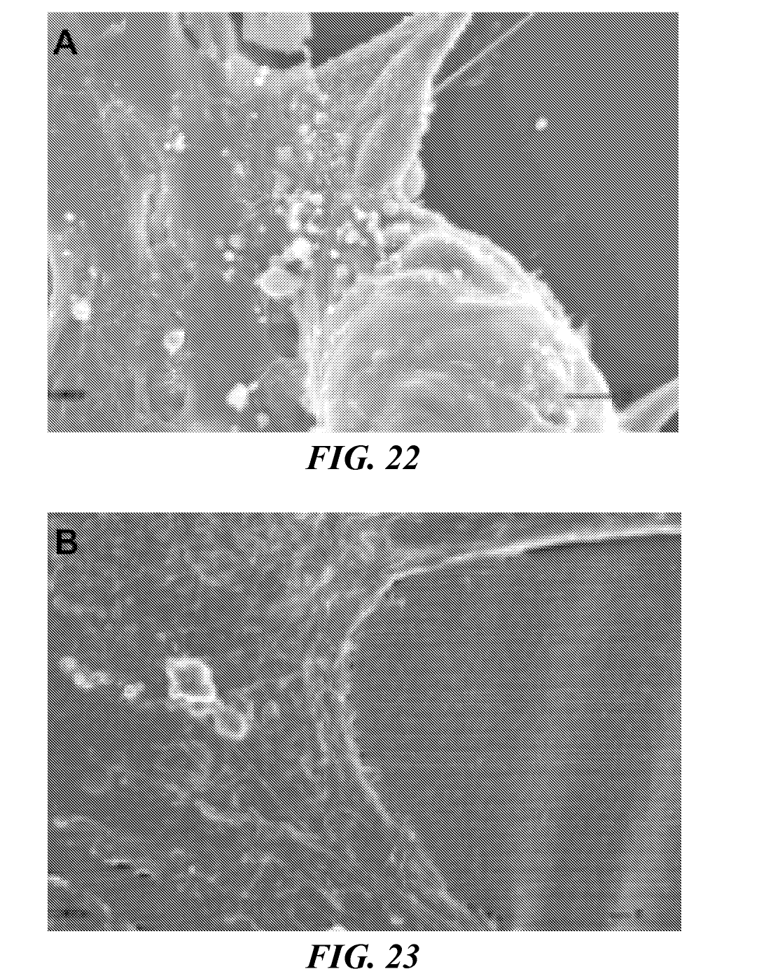

FIGS. 22 and 23, respectively, are SEM micrographs of sheets of fibroblasts cultured on a blank and a nano-patterned surface. Both sheets of fibroblasts tended to detach from the surface if disturbed, but the fibroblast sheets detached more readily on the blank surface and had greater random orientations of the cells compared to the patterned surfaces. FIG. 23 is magnified to show the nano-patterned surface.

Results and Conclusions: We have detected biofilms on silicone surfaces associated with implant material removed from the body due to recurrent infection at the site of the implant. We developed a molding system to introduce micro-patterns and nano-patterns into implant material thereby tailoring the surface energies and other surface properties. Using a culture-model to grow biofilms similar to that seen on the implant material, we determined micro-patterned and nano-patterned silicone surfaces can modify biofilm organization. In addition, we found those patterns effect fibroblast attachment, migration and organization in a manner that suggests they will improve wound healing adjacent to the implant.

Manipulation of a silicone surface by micro-patterning and nano-patterning affects the formation of bio-films and the growth/interaction of fibroblasts. Patterned silicone was exposed to a 24 hour culture of a S. aureus strain known to robustly form bio-film. All surfaces tested grew bio-films. All features such as pattern depth, pattern periodicity and fill factor are seen to affect bio-film development. When the groove valleys were sufficiently far apart (larger than the bacteria), the bio-film was contained within the grooves and did not tend to extend across the grooves. Bio-film formation on bare or nano-patterned surfaces grew in random patterns. The nano-patterned surfaces grew the least dense biofilm structures compared to unpatterned surfaces.

Similarly prepared patterned silicone surfaces were exposed to human foreskin fibroblasts. The fibroblasts were affected by the patterning such that they grew along the pattern. When the culture period was extended, the sheet of fibroblasts on patterned surfaces formed organized layers that stayed intact better than the fibroblast sheets grown on the blank silicone surface. Fibroblasts grown on blank silicone tended to detach, contract and mound-up more often than those grown on the patterned surfaces. This result is expected to be reflected in fibroblast interaction with the implant surface and scar formation.

Micro-patterning and nano-patterning the surface with allow for tailoring the surface energies in order to guide electrode insertion, for example a cochlear implant electrode or a deep brain electrode, in order to position the electrode without damaging surrounding tissues.

One of skill in the art will readily appreciate the scope of the invention from the foregoing and the claims here below, and that the invention includes all disclosed embodiments, modifications of these that are obvious to a person of skill in the art, and the equivalents of all embodiments and modifications, as defined by law.

1. A medical implant comprising implant surfaces exposed to body tissue when the medical implant is inserted into a body of a recipient, the medical implant comprising a nano-sized pattern on at least a portion of the implant surfaces.

2. The medical implant of claim 1, wherein the nano-sized pattern comprises a periodic pattern.

3. The medical implant of claim 1, wherein the nano-sized pattern comprises geometric features

4. The medical implant of claim 1, wherein the nano-sized pattern comprises grooves.

5. The medical implant of claim 1, wherein the nano-sized pattern includes grooves, the grooves grouped into sets of grooves, the sets of grooves spaced at predetermined intervals.

6. The medical implant of claim 1, wherein the nano-sized pattern has a characteristic dimension in the size range from about 1 nm to about 400 nm.

7. The medical implant of claim 1, wherein the nano-sized pattern comprises any of circles, triangles, rectangles, pentagons and hexagons.

8. The medical implant of claim 1, wherein the implant surfaces of the medical implant comprises silicone.

9. The medical implant of claim 1 wherein the nano-sized pattern comprises a pattern imposed upon implant surfaces prior to cure of the silicone surfaces.

10. A medical implant comprising implant surfaces exposed to body tissue when the medical implant is inserted into a body of a recipient, the medical implant comprising a nano-sized pattern on at least a portion of the implant surfaces, the pattern modifying micro-organism and/or fibroblast adhesion to the implant surfaces.

11. The medical implant of claim 10, wherein the nano-sized pattern comprises geometric features.

12. The medical implant of claim 11, wherein the geometric features includes parallel grooves.

13. The medical implant of claim 12, wherein the grooves, at least initially upon implantation of the medical implant into a recipient, modify fibroblast adhesion to the implant surfaces such that fibroblasts tend to preferentially align substantially parallel to the grooves.

14. A medical implant comprising a micro- or nano-sized pattern on at least a portion of the implant surfaces, wherein the grooves, at least initially upon implantation of the medical implant into a recipient, modify micro-organism adhesion to the implant surfaces such that micro-organisms tend to collect primarily or along the grooves.

15. The medical implant of claim 10, wherein the nano-sized pattern is periodic.

16. A medical implant comprising a biocompatible material, the medical implant having surfaces exposed to body tissue and fluid when the medical implant is surgically implanted into a body of a recipient, the surfaces comprising on at least a portion thereof a nano-sized pattern.

17. The medical implant of claim 16, wherein the nano-sized pattern comprises a biocompatible material different from the biocompatible material of the medical implant.

18. The medical implant of claim 16, wherein the nano-sized pattern comprises silicone.

19. The medical implant of claim 16, wherein the medical implant is selected from knee implants, hip implants, cardiac stents, cochlear implants, deep brain electrodes, catheters, pacemakers, breast implants, and tympanostomy tubes.

20. The medical implant of claim 16, wherein the medical implant has an altered surface energy, due to micro-patterning or nano-patterning.

21. A medical implant comprising implant surfaces exposed to body tissue when the medical implant is inserted into a body of a recipient, the medical implant comprising a nano-sized pattern superimposed over one or more surfaces of a micro-sized pattern on at least a portion of the implant surfaces.
